(19) United States
(12) Patent Application Publication
(10) Pub. No.: US 2007/0016168 A1
(43) Pub. Date: Jan. 18, 2007

(54) Method of making a catheter device

(76) Inventor: Anthony J. Conway, Chatfield, MN (US)

Correspondence Address: MERCHANT & GOULD PC, P.O. BOX 2903, MINNEAPOLIS, MN, 55402-0903, US

(21) Appl. No.: 11/170,193

(22) Filed: Jun. 29, 2005

(51) Int. Cl. A61M 27/00

(52) U.S. Cl. 604/544; 604/102.01; 427/2.1; 264/678; 264/512

(57) ABSTRACT

The invention relates to a method of manufacturing a Foley-type catheter. The catheter includes a silicone catheter shaft having a retention balloon. One method of manufacturing the Foley-type catheter includes immersing the silicone retention balloon in mineral oil.

### FIELD OF THE INVENTION

The invention relates to a Foley-type catheter having a retention balloon. More particularly, the invention relates to a method of making a catheter having a silicone rubber retention balloon.

### BACKGROUND OF THE INVENTION

Foley-type catheters are tube-like devices that are used to drain urine from a patient's bladder. Foley catheters are inserted through the urethra and typically held in place with an inflatable balloon. The balloon is in a deflated position when the catheter is first inserted. Then, once the catheter is in the proper position, the balloon is inflated with a fluid. The inflated balloon is larger in diameter than the diameter of the urethra and thereby physically inhibits movement of the catheter. Foley catheters are also known as “indwelling” catheters because they are designed to be left in place for a period of time.

Latex rubber is most often used in the manufacture of Foley catheters. However, latex rubber can be problematic as many patients have latex allergies. To provide an alternative for patients with allergies, silicone rubber has since been used to make Foley catheters. Silicone rubber does not, however, have the same elastic properties as latex rubber. As a result, balloons of Foley catheters made with silicone rubber can exhibit “cuffing.”

Cuffing refers to the situation in which the balloon tends to fold over on itself or shift toward the bladder end of the catheter. Because the balloon is attached at its end to the shaft of the catheter, a cuff forms when the outer expanded portion of the balloon pushes over the inner attached end of the balloon. This cuff can remain when the balloon is deflated before withdrawal of the catheter from the patient. The cuff results in the deflated balloon having a larger diameter than it did when it was first inserted. The increased diameter can result in discomfort and injury to patients. Accordingly, a need exists for a silicone rubber Foley catheter that resists cuffing.

### SUMMARY OF THE INVENTION

One aspect of the present disclosure relates to a catheter having a retention balloon constructed of a material saturated with oil. Other aspects of the present disclosure relates to methods of manufacturing a catheter. One method includes providing a balloon catheter having a retention balloon, and saturating the retention balloon of the balloon catheter shaft with oil. Another method includes providing a balloon catheter having a retention balloon, and immersing the retention balloon in an oil bath.

A variety of examples of desirable product features or methods are set forth in part in the description that follows, and in part will be apparent from the description, or may be learned by practicing various aspects of the disclosure. The aspects of the disclosure may relate to individual features as well as combinations of features. It is to be understood that both the foregoing general description and the following detailed description are explanatory only, and are not restrictive of the claimed invention.

### BRIEF DESCRIPTION OF THE FIGURES

FIG. 1A is a schematic view of a catheter is an original deflated configuration;

FIG. 1B is a schematic view of a catheter in an inflated position wherein the balloon is cuffing;

FIG. 1C is a schematic view of a catheter in a deflated position wherein the balloon has retained a cuff;

FIG. 2 is a partial cross-sectional view of an embodiment of a Foley catheter made in accordance with the present disclosure;

FIG. 3 shows a cross-sectional view of an embodiment of a Foley catheter retention balloon formed with ribs;

FIG. 4 is a partial cross-sectional view of an extruded double lumen tube of the Foley catheter of FIG. 2;

FIG. 5 is a cross-sectional view of the extruded double lumen tube of FIG. 4, as shown from line 202-202′;

FIG. 6 is a partial cross-sectional view of the tube shown in FIG. 4 after an opening is formed in an outer surface;

FIG. 7 is a cross-sectional view of the tube of FIG. 6, as shown from line 204-204′;

FIG. 8 is a partial cross-sectional view of the double lumen tube shown in FIG. 6 after a portion of a capillary lumen has been filled with a polymeric bonding composition;

FIG. 9 is a cross-sectional view of the tube of FIG. 8, as shown from line 206-206′;

FIG. 10 is a partial cross-sectional view of the double lumen tube shown in FIG. 8 after a tip is affixed to a distal end of the tube;

FIG. 11 is a schematic view of a portion of a rack used to retain a plurality of tubes during manufacture of a plurality of Foley catheters;

FIG. 12 is a partial cross-sectional view of an intermediate tube similar to the tube shown in FIG. 10 at an intermediate stage of manufacture;

FIG. 13 is a partial cross-sectional view of an intermediate tube similar to that shown in FIG. 12, but following a first dipping step wherein the outer surface is coated with a bond preventing agent;

FIG. 14 is a cross-sectional view of the intermediate tube of FIG. 13, as shown from line 211-211′;

FIG. 15 is a partial cross-sectional view of an intermediate tube similar to that shown in FIG. 13, but after a subsequent dipping step or steps in which a portion of the coating of bond preventing agent has been removed;

FIG. 16 is a partial cross-sectional view of an intermediate tube similar to that shown in FIG. 15, but shown after formation of a balloon layer;

FIG. 17 is a partial cross-sectional view of an intermediate tube similar to that shown in FIG. 16, but shown after formation of a sheath layer;

FIG. 18 is a partial cross-sectional view of a portion of an embodiment of a Foley catheter having a finish layer; and

FIG. 19 is a schematic illustration of an apparatus used to automate the production of Foley catheters in accordance with the present invention.

### DETAILED DESCRIPTION OF THE INVENTION

Cuffing

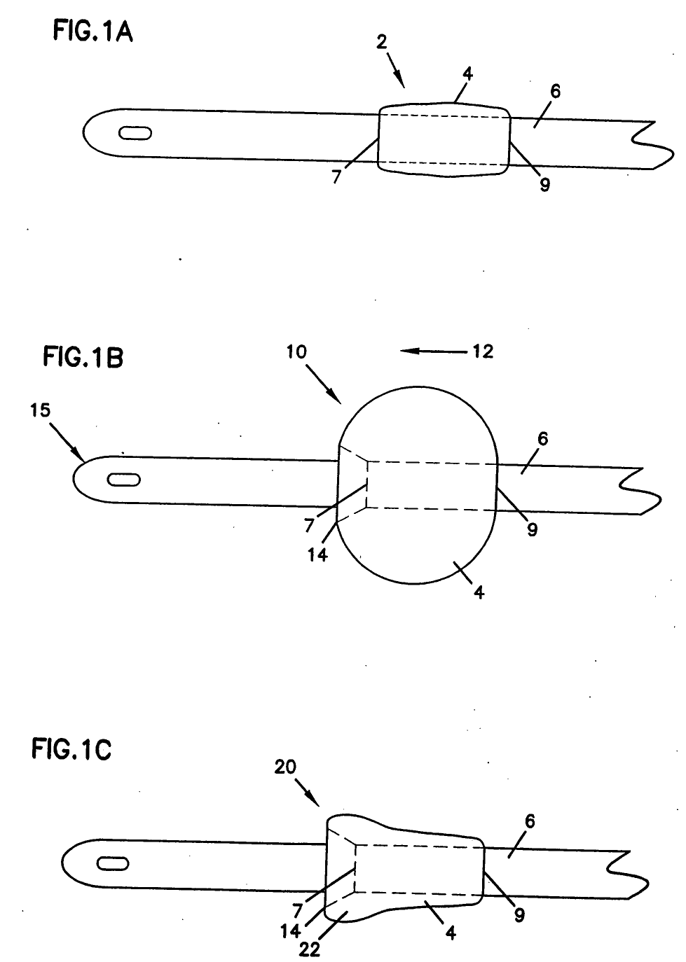

As described above, balloon catheters made with silicone rubber can exhibit problematic cuffing. FIG. 1A shows a schematic view of a catheter in a deflated configuration 2. The catheter includes a balloon 4 and a catheter shaft 6. In the deflated configuration 2, the balloon 4 does not overlap either its distal end 7 or its proximal end 9. Further, in the configuration shown in FIG. 1A, the balloon 4 adds only a small increment to the diameter of the catheter shaft 6 because of how the un-inflated balloon 4 lies flat over the catheter shaft 6.

However, as described above, balloon catheters made with silicone rubber may exhibit problems with cuffing. FIG. 1B is a schematic view of a catheter in an inflated configuration 10 wherein the balloon 4 is cuffing. Cuffing refers to the situation in which the balloon 4 tends to be shifted toward the bladder end 15 of the catheter (in the direction of arrow 12) forming a cuff 14, as the balloon 4 itself is pressed against the bladder wall when holding the catheter in place. Since the balloon 4 is attached at its distal end 7 to the catheter shaft 6, the balloon forms a cuff 14 as the outer expanded portion of the balloon 4 is pushed over the inner attached distal end 7 of the balloon 4.

The cuff 14 that forms tends to remain when the balloon 4 is deflated. FIG. 1C is a schematic view of a catheter in a deflated configuration 20 after having been inflated wherein the balloon formed a cuff 14. The cuff 14 results in the deflated balloon 4 having a larger diameter in an area 22 of the balloon 4 over the cuff 14 than when first inserted. A balloon that has cuffed may be 12 French sizes larger at the cuff, for example, than the actual catheter shaft 6. The increased diameter can result in discomfort and injury to patients.

Method of Making Cuff Resistant Catheters

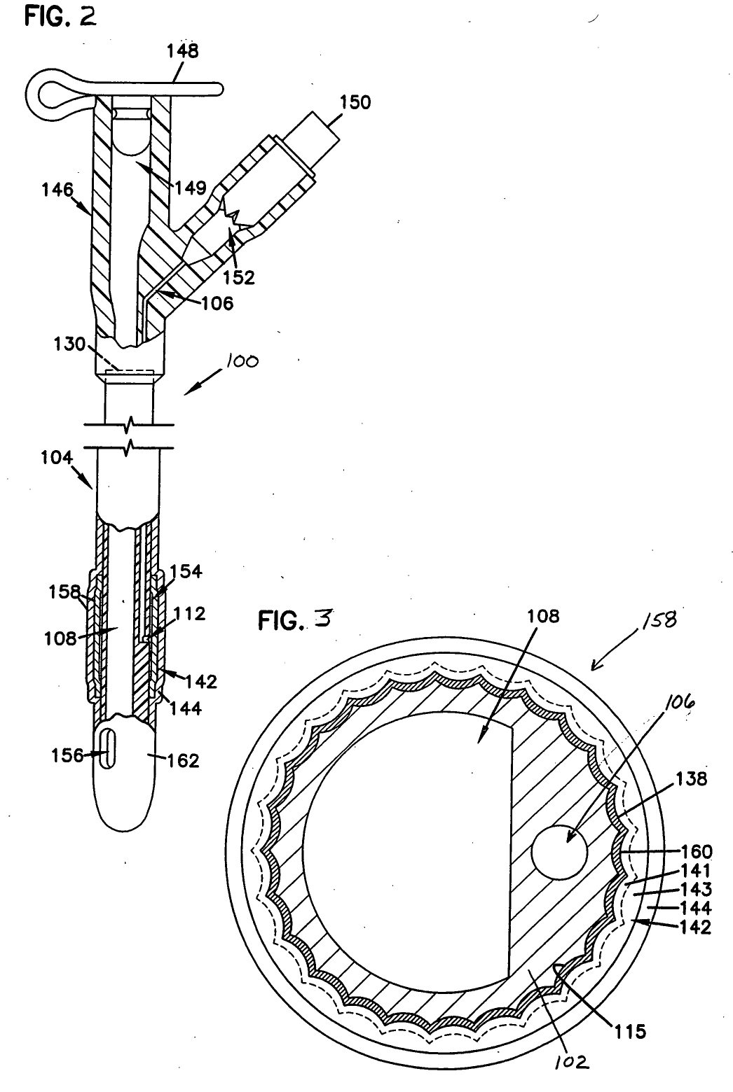

Referring now to FIG. 2, one embodiment of a Foley catheter 100 manufactured in accordance with the present disclosure is illustrated. The Foley catheter 100 includes a catheter shaft 104 and an end piece 146. The catheter shaft 104 includes a retention balloon 158 having a balloon cavity 154. Further details of the components of such Foley catheters 100 are described and disclosed in U.S. patent application Ser. No. ______ (having Attorney Docket No. 8740.106USI1), and U.S. patent application Ser. No. 11/039,074; which applications are incorporated herein by reference.

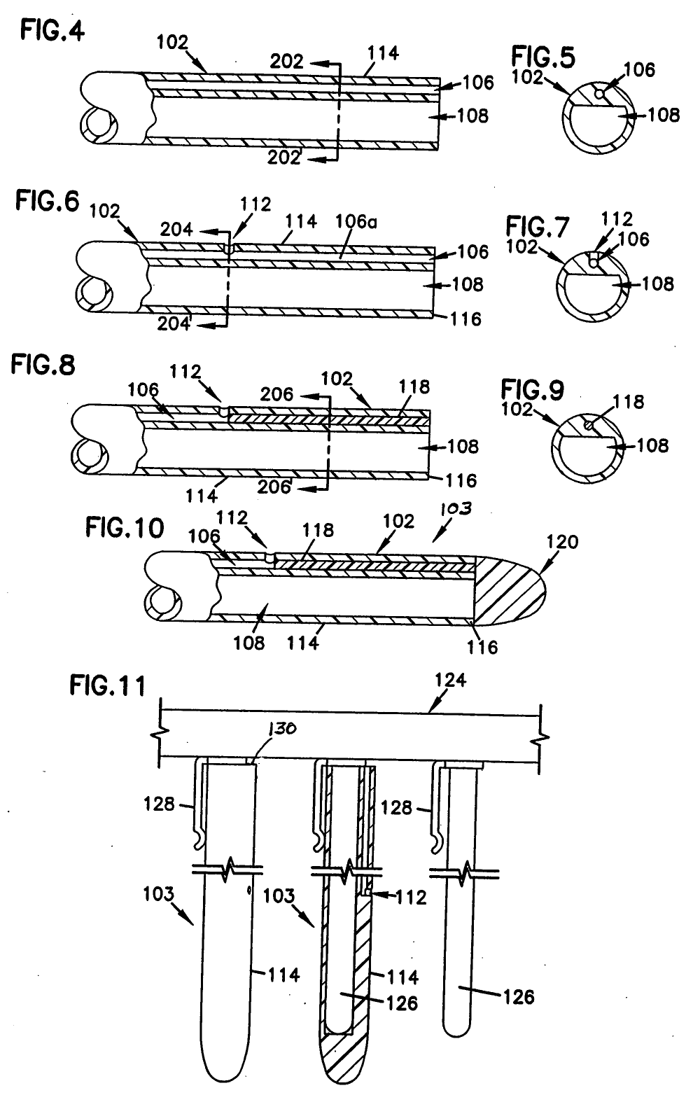

Referring now to FIGS. 4 and 5, the catheter shaft 104 (FIG. 2) of the Foley catheter 100 is constructed from a double lumen tube 102. The double lumen tube 102 is typically extruded, however, the double lumen tube can be made by any known process that yields a double lumen tube construction. The double lumen tube 102 defines a capillary lumen 106 and a fluid conduit lumen 108. Typically, the double lumen tube 102 is made of a resilient polymeric material. In one embodiment, the polymeric material is a biocompatible polymeric material, such as silicone rubber, for example.

The double lumen tube 102 is cut to a desired length. Referring to FIGS. 6 and 7, a capillary lumen access opening 112 is created in an outer surface 114 of the double lumen tube 102. The capillary lumen access opening 112 communicates with the capillary lumen 106.

Referring now to FIGS. 8-10, an intermediate tube 103 (FIG. 10) is prepared from the double lumen tube 102 shown in FIG. 6. In preparing the intermediate tube 103, a measured amount of a filling composition or polymeric bonding composition 118, such as silicone rubber or another suitable polymeric bonding material, is injected into a portion 106a (FIG. 6) the capillary lumen 106 from a distal end 116 of the double lumen tube 102. The capillary lumen portion 106a is filled with the filling composition 118 up to a point just below the capillary lumen access opening 112.

A tip 120, such as a rounded silicone rubber tip, is affixed to the distal end 116 of the tube 102. One method of affixing the tip 120 to the tube 102 includes inserting the distal end 116 of the tube 102 into a molding apparatus (not shown) to mold the tip 120 on the end of the tube 102. Other methods of affixing the tip 120 can be employed.

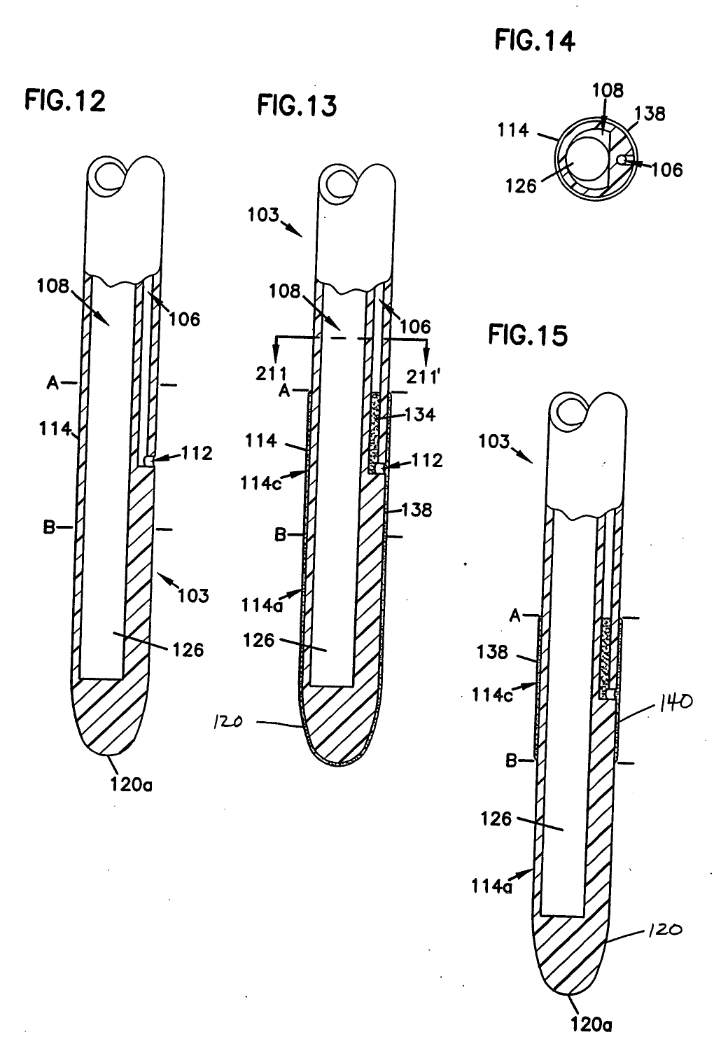

In one embodiment of the present method, the intermediate tube 103 (FIG. 10) is made entirely of silicone rubber. For example, the tip 120 and the filling composition 118 of the intermediate tube 103 are of the same material (silicone rubber) as the double lumen tube 102. Therefore, the tip 120 and the filling composition 118 form integral portions of the intermediate tube 103. FIGS. 12-17 show the intermediate tube 103 as an integral polymeric unit made of a single material.

Preferably the process of manufacturing the catheter 100 is an automated process. One of skill in the art will appreciate that while the methods are described as practiced in an automated fashion, the methods can also be practiced in a non-automated or manual, hand-performed fashion, or a semi-automated fashion.

The automated process involves securing a plurality of the intermediate tubes 103 to a rack or pallet 124, as shown in FIG. 11. The pallet 124 includes a plurality of support rods 126 so that entire sets of catheters 100 can be manufactured simultaneously. In one embodiment, the pallet 124 has 400 spring steel support rods 126 attached to the pallet 124 in a 20-by-20 configuration. Each of the rods 126 is about 1 inch from adjacent rods.

Referring still to FIG. 11, each of the support rods 126 is equipped with a retaining clip 128. The intermediate tubes 103 are secured on the support rods 126 by positioning the individual support rods 126 within the fluid conduit lumens 108 (FIG. 10) of the intermediate tubes, and sliding the intermediate tubes 103 up over the support rods 126. Each of the intermediate tubes 103 is typically positioned on the support rod 126 such that a proximal end 130 of the intermediate tube 103 abuts against the base of the retaining clips 128, or such that the tip 120 of the intermediate tube 103 fits snugly against the distal tip of the support rod 126. Although not shown, it is believed that the intermediate tubes 103 can be secured on the support rods 126 without the aid of the retaining clips 128. This is because extruded double lumen tubes 102 generally have a slight bend. This permits the intermediate tube 103 to be secured on the support rod 126 via a friction fit without the aid of the clip 128.

FIG. 19 schematically illustrates the pallet 124 loaded with the plurality of intermediate tubes 103. The pallet 124 transfers the intermediate tubes 103 from place to place via a transporting mechanism 122. For example, the transporting mechanism 122 moves or transfers the loaded pallet 124 between a series of baths or dip tanks used to manufacture the completed Foley catheter 100 shown in FIG. 2. The series of dip tanks are used to form the catheter shaft 104 having the retention balloon 158 of the Foley catheter 100.

In particular, after the intermediate tubes 103 loaded on the pallet 124, the intermediate tubes 103 are transported to a first bath or dip tank 133 by the transporting mechanism 122 (FIG. 19). The first dip tank 133 is raised so that all of the intermediate tubes 103 are simultaneously coated with a bond preventing agent; preferably, a removable bond preventing agent. While the present method relates to machinery that raises and lowers the dip tanks relative to the pallet 124, it is contemplated that the pallet 124 can also be lowered and raised relative the dip tanks.

Still referring to FIG. 19, the intermediate tubes 103 are immersed or dipped into the first dip tank 133 containing the bath of the removable bond preventing agent. The removable bond preventing agent includes materials that form a semi-solid film or coating on surfaces when cooled or dried. Examples of such materials include petroleum jelly or petrolatum, other oil base substances that form a semi-solid film upon cooling to room temperature, liquid soaps that dry to form a semi-solid film, aqueous soap or detergent solutions, aqueous or oil based film forming materials, and the like. In one method, hot petrolatum is used, and in another method, a liquid soap, such as LIQUID IVORY® soap from Proctor & Gamble, Cincinnati, Ohio, is used.

Referring now to FIG. 13, the intermediate tubes 103 are immersed in the first dip tank 133 to a desired level designated by line A. Immersing the intermediate tubes 103 into the first bath 133 coats the outer surface 114 of the intermediate tube 103 with the removable bond preventing agent. In addition, the agent enters the capillary lumen access opening 112 and runs up into the capillary lumen 106 (as shown in FIG. 13). In one embodiment the agent is petrolatum, heated to about 140°-160° F., typically about 150° F. At this temperature, the petrolatum runs up into the capillary lumen 106 through the capillary lumen access opening 112 with the assistance of the “capillary effect”, which draws the fluid into the capillary lumen 106 to the level 133a (FIG. 19) of the petrolatum in the first dip tank 133. As the intermediate tubes 103 are withdrawn from the hot petrolatum, petrolatum on each of the tubes 103 cools and solidifies to form a semi-solid bond preventing coating 138 (FIG. 13) on the outer surface 114. Likewise, a semi-solid filling 134 in the capillary lumen 106 and the capillary lumen access opening 112 is created, which cooperate to plug the capillary lumen access opening 112.

In an alternate embodiment, the bond preventing agent in the first dip tank 133 is liquid soap. The liquid soap is typically at a room temperature (about 62′-74° F.). When the tubes 103 are withdrawn from the first dip tank of liquid soap, the soap dries to form the bond preventing coating 138, just as the hot petrolatum did when cooled. Although both of these bond preventing agents are effective, there is some advantage to using liquid soap. Liquid soap does not require the added expense of providing a heated dip tank. Further, in certain embodiments, soap is easier to remove from the capillary lumen 106 and the subsequently formed balloon cavity 154 (FIG. 2).

After the outer surface 114 of the intermediate tubes 103 is coated and the capillary lumen 106 and the capillary lumen access openings 112 are plugged with the bond preventing agent, the intermediate tubes 103 are dipped in a series of dip tanks provided to remove a portion of the bond preventing coating 138. As shown in FIGS. 13 and 15, the coating 138 is removed from a portion 114a of the outer surface 114 below the line designated B. In one method, for example, the step of removing the portion of bond preventing coating 138 includes dipping the intermediate tubes 103 in series of different dip tanks.

In particular, one method includes advancing and positioning the pallet 124 at a second dip tank 135 (FIG. 19) containing white USP petrolatum heated to about 250° F. The intermediate tubes 103 are immersed into the super-heated petrolatum to a level designated by line B in FIGS. 13 and 15. The super-heated petrolatum contacts the coating 138 on outer surface 114 of the intermediate tubes 103 to largely remove the coating 138 from the outer surface portion 114a of the intermediate tubes 103. The bond preventing coating 138 is removed from a location where the distal end of the retention balloon 158 will be located (designated by line B) to the distal end 120a of the tip 120 of the intermediate tubes 103. Some residual petrolatum may remain on the outer surface portion 114a; however, most of the petrolatum is removed.

Referring to FIG. 19, the pallet 124 then advances to a third dip tank 137 containing mineral spirits heated to about 200° F. The intermediate tubes 103 are immerse into the mineral spirits to the same depth as they were immersed in the super-heated petrolatum in the second dip tank 135. The mineral spirits remove all but a trace amount of the bond preventing coating 138 from the outer surface portion 114a of the intermediate tube 103.

Last, the pallet 124 moves to a fourth dip tank 139 containing a volatile organic solvent such as toluene, trichloroethane or the like. The intermediate tubes 103 are immersed in the fourth tank 139 to the same depth as previously immersed in the second and third tanks 135 and 137. The organic solvent removes essentially all traces of the coating 138 from the outer surface portion 114a of the intermediate tube 103. As shown in FIG. 15, the intermediate tube 103 now has a band 140 of the bone preventing coating 138 located around the axial circumference of the intermediate tube 103. The band 140 is located along a portion 114c of the outer surface 114 where the retention balloon 158 and the balloon cavity 154 are subsequently formed.

After the outer surface portion 114a of the intermediate tube 103 is substantially stripped of the bond preventing coating 138, the intermediate tubes 103 are dipped in a polymeric bonding composition, such as silicone rubber. In one method, the pallet 124 advances to a fifth dip tank 141 containing a heptane dispersed solution of silicone rubber (such as Dow Corning C6-515 or another appropriate balloon compound).

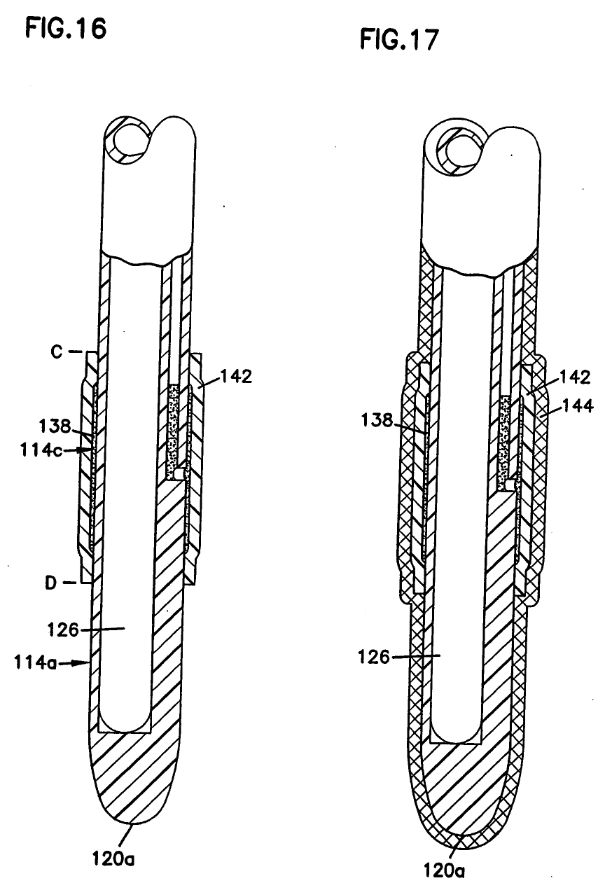

The intermediate tubes 103 are immersed in the fifth dip tank 141 so that the silicone rubber covers and extends the length of intermediate tube 103 up to line C shown in FIG. 16. In some embodiments, line C is about 0.25 inches above the top of the band 140 of the bond preventing coating 138. This deposition process can be repeated until a balloon layer 142 having a desired diameter relative to a predetermined diameter of the catheter shaft 104 is formed. In one embodiment, the difference in diameters is less than or equal to about 4 French sizes (e.g., about 0.052 inch), for example, no more than 4 French sizes (0.052 inch).

As shown in FIG. 16, the balloon layer 142 does not extend along the entire length of the intermediate tube 103. Rather, the intermediate tubes 103 are dipped in a solvent to remove a portion of the silicone rubber located below line D of FIG. 16. In some embodiments, line D is about 0.25 inches below the band 140 of bond preventing coating 138. The resulting layer is the balloon layer 142 of the Foley catheter 100. Referring to FIG. 19, removing the portion of silicone rubber involves advancing the pallet 124 to a sixth dip tank 143 containing a solvent effective to remove the deposited silicone rubber. Suitable solvents include xylene or toluene.

At this point, the intermediate tubes 103 can be air dried for approximately 30 minutes to remove or evaporate solvents from the balloon layer 142. In addition, the balloon layer 142 of the tubes 103 can be cured before further processing; however, in some methods, the curing can be delayed until later in the processing. One of skill in the art will appreciate that there are many methods of curing silicone rubber. By way of example, the silicone rubber can be cured through a heat cure step for approximately two hours at a temperature just below the boiling point of any solvent used in any of the silicone rubber dip solutions.

Referring now to FIG. 3, in one embodiment, the Foley catheter 100 includes a plurality of ribs 160 formed in the retention balloon 158 of the catheter shaft 104. In this embodiment, the extruded double lumen tube 102 includes a series of generally parallel grooves 115 (e.g., undulations or channels). Typically, the grooves 115 extend parallel with the longitudinal axis of the tube 102. When grooves 115 are provided I the tube 102, the ribs 160 inherently form on an inner surface of the retention balloon 158 (i.e., a first region 141 of the balloon layer 142). In particular, the ribs 160 form because the bond preventing coating 138 follows the grooves 115 in the outer surface 114 of the tube (102, 103). The balloon layer 142 also then follows the grooves and provides a structure (i.e. the rib 160) that is an inverse of the groove 115.

In some embodiments, the ribs 160 are made of a silicone rubber having different properties than the silicone rubber used for the remainder of the retention balloon 158. For example, the silicone rubber used to make the ribs 160 can be less pliable than the silicone rubber used to form the remainder of the balloon layer 142. A less pliable silicone rubber can include, for example, a higher modulus silicone such as a 50/50 mixture of Dow Corning Q7-4850 and Dow Corning Q7-4720. The less pliable silicone rubber defines the first region 141 of the balloon layer 142 having the ribs 160. Thereafter, the remaining balloon layer 142, i.e., the second region 143, can be formed with more pliable silicone rubber. The less pliable material and/or the thickened structures (i.e., the ribs 160) of the balloon layer 142 aids in reducing the likelihood of cuffing. In addition, while not intending to be bound by theory, it is believed that by creating ribs 160 in a direction parallel to the catheter shaft 104, stretching of the balloon in that direction is limited, to further resist longitudinal balloon shifting or cuffing.

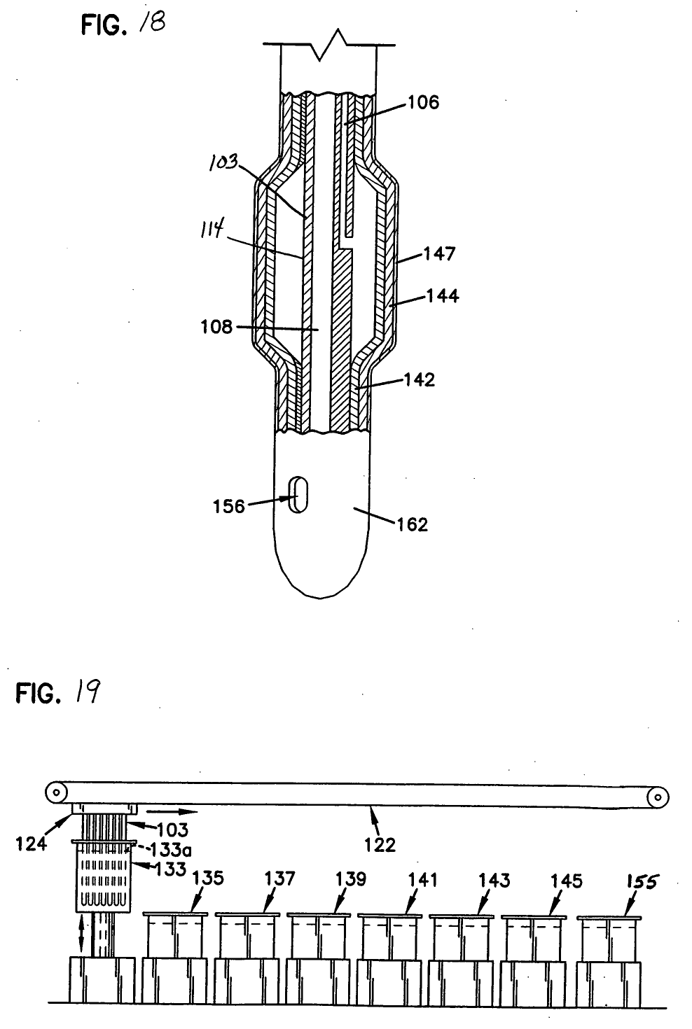

Referring now to FIG. 17, after the balloon layer 142 has been formed, a substantial majority of the intermediate tube 103 is immersed into a heptane dispersed solution of silicone rubber (such as Dow Corning C6-515 or another appropriate balloon compound) to form a sheath layer 144. In particular, the pallet 124 moves to a seventh dip tank 145 (FIG. 19) containing the solution of silicone rubber. The intermediate tubes 103 are immersed into the seventh tank 145 as many times as is necessary to obtain the desired sheath layer thickness. The sheath layer 144 is then allowed to air dry for a period of about 30 minutes. By forming the sheath layer 144 along the entire length of the intermediate tube 103, the retention balloon 158 is thickened, but the difference in thickness between the retention balloon 158 and the catheter shaft 104 is maintained.

Optionally, the pallet 124 can be advanced to an eighth dip tank (not shown) containing a thin finish-type silicone rubber (such as Dow Corning 4720). The intermediate tubes 103 are dipped in the finish-type silicone rubber to create a finish layer 147 (FIG. 18). The finish layer 147 provides beneficial tactile properties to the exterior of the catheter shaft 104 of the Foley catheter 100.

The balloon layer 142, the sheath layer 144, and the optional finish layer 147 formed on the intermediate tube 103 now define the catheter shaft 104. The catheter shaft 104 is typically allowed to air dry to permit solvents in the balloon layer 142 and the sheath layer 144 to evaporate. Typically, the shaft 104 is dried, and subsequently cured, at an elevated temperature. In one method, the catheter shafts 104 are permitted to dry for approximately two hours, and then are heat cured for an additional two hours. The heat curing process includes exposing the catheter shafts 104 to a temperature chamber at about 200° F. Care is taken to keep the curing temperature below the boiling temperatures of the solvent so as to prevent unsightly bubbling of the solvent within the balloon layer 142 and the sheath layer 144. One of skill in the art will appreciate that the drying time and the curing time and temperature are approximate and can be varied depending on the specific materials and solvents used.

After the catheter shaft 104 is dried, cured, and cooled, the catheter shaft 104 is immersed in oil. One feature of the present disclosure relates to the method of manufacturing the disclosed Foley catheter 100, including the step of dwelling or immersing the catheter shaft 104 in oil. Immersing or soaking the catheter shaft 104 in oil decreases the occurrence of balloon pruning and cuffing by enhancing the elasticity qualities of the silicone retention balloon 158. Conventionally manufactured silicone catheter products tend to elastically breakdown when exposed to urine or other bodily fluids, such as stomach acid or digestive fluids. The elastic breakdown of silicone causes a loss of material memory, resulting in pruning and cuffing. When the retention balloon 158 of the present catheter shaft 104 soaks in oil, oil fills the pores of the silicone material. The oil-saturated silicone prevents urine from otherwise filling the pores and thereby reduces elastic breakdown. The oil bath increases memory or return of the balloon, and lessens pruning and cuffing.

Referring to FIG. 19, in the oil-dwelling manufacturing step, the pallet 124 moves to a ninth dip tank 155 containing oil. In one method, the oil is mineral oil, such as Holland Drake Oil No. 7 or No. 9, for example. The catheter shaft 104 soaks or dwells within the tank 155 of oil for a period of time, up to 72 hours, typically about 24 hours. Typically, the oil is at room temperature. In alternative methods, the oil can be heated, for example, to about 200° F., to speed up the absorption of oil and reduce the dwell time. Other types of oils and other immersion periods can be employed to impregnate and saturate the silicone catheter construction with oil.

To complete the Foley catheter 100 as shown in FIG. 2, the end piece 146 is secured to the proximal end 130 of the catheter shaft 104. The end piece 146 can include a cap 148 for closing a first proximal opening 149 to the fluid conduit lumen 108. In the illustrated embodiment, the end piece 146 is equipped with a luer valve 150 for engagement in and closure of a second proximal opening 152 communicating with the capillary lumen 106. The completed Foley catheter 100 also includes a drainage eye or fluid conduit access opening 156 formed in an exterior surface 162 of the catheter shaft 104. The drainage eye 156 is in fluid communication with the fluid conduit lumen 108.

In one method of manufacture, the end piece 146 is made by a process of injection molding. In particular, the proximal end 130 of the balloon catheter shaft 104 is inserted into an injection molding apparatus after the balloon layer 142 and the sheath layer 144 have been cured. A polymeric bonding composition, such as silicone rubber, is then injected into the mold (not shown) and the end piece 146 is molded onto the proximal end 130 of the balloon catheter shaft 104 to make the completed Foley catheter 100 shown in FIG. 2.

In an alternative method, the end piece 146 is molded to the proximal end 130 of the double lumen tube 102 prior to the automated process of immersing the intermediate tube 103. In this alternative method, the double lumen tube 102 is inserted into the injection molding apparatus, the polymeric bonding composition is then injected into the mold, and the end piece 146 is molded onto the double lumen tube 102. The intermediate tube 103 is then constructed. Subsequently, the first proximal opening 149 of the end piece 146 is secured to the support rod 126 by the retaining clip 128. The intermediate tube 103 is then dipped in the series of baths or dip tanks as previously described.

Referring now to FIG. 18, the retention balloon 158 of the Foley catheter 100, which includes the balloon layer 142 and the sheath layer 144, does not bond to the outer surface 114 of the intermediate tube 103. The retention balloon 158 is free to expand or inflate due to the bond preventing coating 138 that remained on the outer surface portion 114c (FIGS. 13 and 15) of the intermediate tube 103 during manufacture.

When a fluid is pumped or injected into the capillary access lumen 106 of the Foley catheter 100, the retention balloon 158 and the balloon cavity 154 expand. Any of a variety of known tests can be used to ensure that there are no leaks in the retention balloon 158 of the Foley catheter 100. Typically, a hot aqueous solution is used to test for leaks in the retention balloon 158. The hot aqueous solution also functions to remove the remaining bond preventing coating 138 and filling 134 (FIG. 13) from the balloon cavity 154 and the capillary lumen 106 respectively.

While the present method of manufacturing has been described in the making of a silicone rubber catheter, it is contemplated that the principles of the disclosed method can also be used in the making of a latex catheter. Further, although the present description relates to the making of a silicone rubber catheter, the principles disclosed can also be applied to the making of other silicone rubber devices, such as gastrostomy and other feeding tube devices, suprapubic catheters, and enema cuffs, for example.

The above specification provides a complete description of the. Since many embodiments of the invention can be made without departing from the spirit and scope of the invention, certain aspects of the invention reside in the claims hereinafter appended.

We claim:

1. A method of manufacturing a catheter, the method comprising the steps of:
a) providing a balloon catheter having an inflatable retention balloon; and
b) saturating the inflatable retention balloon with oil.

2. The method of claim 1, wherein the step of saturating the inflatable retention balloon with oil includes saturating the inflatable retention balloon with mineral oil.

3. The method of claim 1, wherein the step of saturating the inflatable retention balloon with oil includes dwelling the inflatable retention balloon in an oil bath for a period of up to 72 hours.

4. The method of claim 3, wherein the step of dwelling the inflatable retention balloon in the oil bath includes dwelling the inflatable retention balloon in the oil bath for about 24 hours.

5. The method of claim 1, wherein the step of saturating the inflatable retention balloon with oil includes immersing the inflatable retention balloon in a heated oil bath.

6. The method of claim 1, wherein the step of providing the catheter includes providing a catheter having an inflatable retention balloon constructed of silicone rubber.

7. The method of claim 1, wherein the step of providing a balloon catheter includes:
a) providing a tube having a first lumen and a second lumen;
b) cutting the tube to a desired length;
c) forming a balloon layer over the tube, the balloon layer having a first end and a second end, each of the first and second ends being attached to the tube; and
d) applying a sheath layer over a portion of the length of the tube.

8. The method of claim 7, further including attaching an end piece to a proximal end of the tube.

9. The method of claim 7, further including creating a drainage eye in an outer surface of the tube that communications with the one of the first and second lumens of the tube.

10. A method of manufacturing a catheter, the method comprising the steps of:
a) providing a balloon catheter having a retention balloon; and
b) immersing the retention balloon of the balloon catheter in an oil bath.

11. The method of claim 10, wherein the step of immersing the retention balloon in the oil bath includes immersing the retention balloon in a mineral oil bath.

12. The method of claim 10, wherein the step of immersing the retention balloon in the oil bath includes immersing the retention balloon for a period of up to 72 hours.

13. The method of claim 12, wherein the step of immersing the retention balloon in the oil bath includes immersing the retention balloon for about 24 hours.

14. The method of claim 10, wherein the step of immersing the retention balloon in the oil bath includes immersing the retention balloon in a heated oil bath.

15. The method of claim 10, wherein the step of providing the catheter includes providing a catheter having a retention balloon constructed of silicone rubber.

16. The method of claim 10, wherein the step of providing a balloon catheter includes:
a) providing a double lumen tube;
b) attaching a tip to a distal end of the double lumen tube;
c) immersing the double lumen tube in a bath of bond preventing agent, and subsequently removing a portion of the bond preventing agent adhered to the double lumen tube;
d) immersing the double lumen tube in a bath of silicone rubber and subsequently removing a portion of the silicone rubber to form a balloon layer; and
e) immersing the double lumen tube in a bath of silicone rubber to form a sheath layer, wherein the retention balloon is defined by the balloon layer and a portion of the sheath layer.

17. The method of claim 16, further including attaching an end piece to a proximal end of the double lumen tube.

18. The method of claim 16, further including creating a drainage eye in an outer surface of the double lumen tube that communications with one lumen of the double lumen tube.

19. The method of claim 16, wherein the step of immersing the retention balloon in the oil bath includes dwelling the retention balloon in the oil bath for a period of time such that oil is absorbed by the portion of the sheath layer and the balloon layer.

20. A catheter, comprising:
a) a catheter shaft; and
b) an inflatable retention balloon attached to the catheter shaft, the inflatable retention balloon being constructed of a material saturated with oil.
